(19) United States
(12) Patent Application Publication
(10) Pub. No.: US 2009/0124928 A1
(43) Pub. Date: May 14, 2009

(54) Apparatus for Circumferential Suction Step Multibiopsy of the Esophagus or Other Luminal Structure with Serial Collection, Storage and Processing of Biopsy Specimens within a Removable Distal Cassette for In Situ Analysis

(76) Inventor: David S. Zimmon, Port Washington, NY (US)

Correspondence Address: COLLARD & ROE, P.C., 1077 NORTHERN BOULEVARD, ROSLYN, NY, 11576, US

(21) Appl. No.: 11/795,615

(22) PCT Filed: Jan. 20, 2006
(86) PCT No.: PCT/US2006/002066

§ 371 Date: Jun. 2, 2008

Related U.S. Application Data
(60) Provisional application No. 60/645,896, filed on Jan. 21, 2005.

(51) Int. Cl. A61B 10/02; A61B 5/00

(52) U.S. Cl. 600/563; 600/565; 600/573

(57) ABSTRACT

A circumferential suction step multibiopsy apparatus for performing a medical procedure has an elongated flexible member with an aperture extending longitudinally therethrough and an actuator positioned within the aperture. The open tube shaft is connected to a side arm for suction or irrigation including radiopaque contrast and dye staining. There is a cassette removably connected to the actuator for cutting and serially collecting biopsy specimens, for in situ chemical, biological or genetic testing by immediate reaction with the biopsy specimens before metabolic changes, degradation or contamination can occur or for fixation, staining and other processing and analysis.

### CROSS-REFERENCE TO RELATED APPLICATIONS

This application claims priority from U.S. Provisional Application No. 60/645,896, filed on Jan. 21, 2005.

### BACKGROUND OF THE INVENTION

1. Field of the Invention

The present invention relates to an apparatus for a circumferential suction step multibiopsy of the esophagus or other luminal structure with serial collection, storage and processing of biopsy specimens in situ. The device simultaneously cuts and captures multiple circumferential suction biopsy specimens of a closely defined size to permit serial entry into a segmented removable distal storage cassette for in situ chemical, biological or genetic testing by immediate contact of reagents with the biopsy specimens before metabolic changes, degradation or contamination can occur or for fixation, staining and other processing and analysis. The cassette may be optically transparent for physical analysis of the tissue without removal from the cassette after separation from the biopsy instrument. Prior to or after biopsy, the open tube shaft with a side arm permits fluid sampling, suction, irrigation, and injection of tissue stains or radiopaque contrast agents at the biopsy site.

2. The Prior Art

It is often necessary to obtain tissue samples for examination from deep within structures. These samples can only be retrieved by catheterization methods using endoscopic or fluoroscopic control, or by blind biopsy. Most biopsy devices used for these techniques removed 1 to a maximum of 4 specimens that were retrieved by removing the biopsy instrument from the patient, separating the biopsy from the sharp biopsy instrument by hand and placing the specimen(s) in a container of fixative solution labeled with the biopsy site and patient identification. During this acquisition and collection process, the minute specimens were frequently lost and are contaminated by passage through the biopsy path or the endoscope instrument channel and by handling. During this process, the staff is exposed to potentially infectious human fluids, tissue and toxic fixatives.

After each biopsy pass and collection, the biopsy instrument was washed to remove fixative and returned to the endoscopist for passage through the endoscope for the next biopsy. This prolonged the procedure and could cause it to fail, if the position of the biopsy instrument could not be reacquired during the repeated passes through the endoscope. This prolongs the procedure, increasing the quantity of sedative administered to the patient, risk and cost.

Furthermore, when the extent of disease such as cancer or dysplasia required diagnosis as in Barrett's esophagus, the precise site of the biopsies was difficult to know with precision. If after processing and microscopy, dysplasia or cancer were later identified, confirmatory biopsy or local excision was difficult as the site of positive biopsy was not well known.

Since the minute pieces could not be easily separated, biopsies obtained from each site were processed in a batch. Often multiple biopsy passes were required because of the limited storage capacity of the biopsy instrument and the need to identify the origin of each biopsy site. Consequently, biopsies from different anatomic sites were handled separately, requiring considerable effort and expense.

The containers for each biopsy level or site were then transported to the pathology laboratory where each container was opened and each specimen batch transferred to individual numbered cassettes that were recorded for later identification. The cassettes were then processed for examination. The processed specimens were then sliced, stained and mounted on labeled slides for microscopic examination. The specimens in each container must be processed and mounted on separate slides to maintain identification and prevent errors in reporting. This was particularly important when the distribution and extent of a cancer was being mapped to determine therapy.

During this complex handling process, small specimens may be lost or damaged. At each stage of handling, the staff is exposed to toxic fixatives and infection from the biopsies. This is particularly true when the unfixed specimen is removed by hand from the sharp biopsy instrument before fixation. The staff is also exposed to allergenic or carcinogenic solvent vapor from the fixative at each transfer step of processing. This tedious, labor intensive process is expensive in staff required, time, equipment and laboratory space.

Specimens needed for chemical, biological or genetic testing require additional biopsies that must be handled separately. These specimens can be contaminated by fluid and tissue in the track traversed to obtain the biopsies and within the channel of the endoscopic instrument. The delay in acquisition and contamination of the specimens limits the accuracy and reliability of the subsequent analysis, particularly when genetic or biological testing is required.

The prior art described in the spring based multipurpose medical instrument in U.S. Pat. No. 5,782,747 to Zimmon, the disclosure of which is herein incorporated by reference, obviates the use of cumbersome metal shafts and coverings to allow use of the shaft lumen for suction or injection.

U.S. Pat. Nos. 5,685,320 and 5,782,747, both to Zimmon, both of which are herein incorporated by reference, describe the use of sharply cut biopsies of closely defined size that are suitable for passage into the lumen of the biopsy device for storage and processing in situ. In U.S. Pat. No. 6,468,227 to Zimmon, herein incorporated by reference, the biopsy device uses a precise distance between the central actuator wire and the cutting blade to control biopsy depth and limits actuator wire movement to control biopsy length. The cut biopsy is then captured within the tube shaft and available to move into the collection cassette. The motive force of suction or fluid pressure propels the precisely cut biopsy from either device into the proximal collection cassette as described in U.S. Pat. No. 6,071,246 to Zimmon, which is herein incorporated by reference.

In U.S. Pat. No. 6,322,522 to Zimmon, which is herein incorporated by reference, the biopsy instrument captures biopsy specimens in a removable cassette or cassettes at the proximal end of the biopsy instrument for immediate processing and analysis without removing and destroying the biopsy instrument to form the cassette. This improvement allows real time specimen analysis during the biopsy procedure and the use of a relatively expensive reusable or reprocessable biopsy instrument.

The serial collection, storage and processing of multiple specimens within a portion of the biopsy instrument yields a great savings of time and effort in processing the biopsies, as well as preventing specimen loss or damage during handling, and protecting staff from infectious material and toxic fixatives. This goal is facilitated by forcing the minute biopsy specimens into a storage cassette of the biopsy instrument and by minimizing the operating parts of the biopsy instrument to maximize the storage volume. Taken together, these improvements greatly reduce the cost of biopsy and processing in the pathology laboratory.

Although the prior art has made safe efficient biopsy deep within the patient possible, a need for additional improvements remains.

### SUMMARY OF THE INVENTION

It is therefore an object of the present invention to provide a circumferential suction step multibiopsy device with serial collection and storage of biopsy specimens in a distal removable biopsy processing cassette that is 15 to 60 French (5 mm to 30 mm) in diameter, that is simple to use and provides precise identification of the biopsy site from acquisition to histopathologic diagnosis when used with a 30 to 220 cm long flexible biopsy instrument.

The device according to the invention comprises a flexible plastic shaft with a relatively large central lumen connected to a side arm for suction or irrigation. A proximal handle connects to the central actuator wire and seals the lumen to permit suction or irrigation. The distal end of the tube shaft attaches to and is sealed to the proximal portion of the biopsy cassette through the shaft lumen to transmit suction and irrigation. The biopsy cassette is comprised of two parts that are stabilized by guide grooves in the metal or plastic cassette to preserve precise alignment and prevent unintended separation of the two parts. The proximal portion has a circumferential blade with a screen positioned above the cutting blade connected to the actuator wire that overlaps the multiple biopsy ports of the distal portion when advanced into the biopsy cassette. The stationary distal biopsy cassette portion has multiple circumferential ports for suction biopsy. Distal to the suction biopsy ports, the cassette is divided into segments so that each side hole is positioned above a separate storage segment.

For biopsy, the instrument is positioned in the lumen by depth using centimeter marks on the instrument shaft and a center line marking the anterior 12 o'clock position. To biopsy, the actuator wire is pulled to retract the blade and open the ports of the distal segment. Suction is applied through the shaft side arm, drawing the surrounding tissue into the side holes. The actuator wire is pushed, forcing the blade over the side holes and cutting the biopsies from the surrounding tissue. The number of biopsies acquired at each step is determined by the number of circumferential biopsy ports in the cassette. The proximal portion screen forces the cut biopsies into individual storage segments. Biopsy orientation, storage and processing may be facilitated by injecting fluid through the side arm.

Each subsequent circumferential step biopsy is performed by moving the biopsy instrument 0.5 to 1.0 cm proximally or distally and repeating the biopsy sequence. Each set of circumferential biopsies is serially collected and stored in the order of acquisition within the storage segments adjacent to each biopsy port until the procedure is completed. At completion, the actuator wire is pushed to position the blade over the biopsy ports and close the cassette. After the instrument is withdrawn, the biopsy cassette is separated from the shaft and the serially stored biopsies processed.

Fluid access for processing is maintained through the shaft lumen and the screen in the proximal portion of the biopsy cassette that prevents mixing or loss of biopsies within the individual storage segments. Additionally, the distal cassette portion may be perforated to allow access of processing fluids and to peel the cassette open after processing. In this case, the perforations are covered with removable plastic to maintain suction during biopsy. Alternatively, the actuator wire may be connected to the distal biopsy segment that is biased open by an actuator wire spring within the cassette. Biopsy is then performed by applying suction and then pulling the actuator wire to move the biopsy ports over the fixed blade and cut the biopsies from the knuckle of tissue extending into the biopsy ports. After completion of the biopsy sequence, the actuator wire is locked in the closed position at the handle to maintain a closed position.

Preferably, the lumen is disposable or reusable after washing and disinfecting. The storage cassette is preferably constructed of material to allow cutting without removal of the biopsies. The cassette is preferably optically transparent, but opaque materials could also be used. Multiple cassettes may be connected to the flexible member.

A serial number identifies each biopsy cassette. A single log prepared at the time of biopsy serves to identify the site, depth and orientation of each biopsy series to the submitter and laboratory for reporting without handling, risk of biopsy loss or documentation error.

Processing for histopathology from fixation to wax embedding is performed with the biopsies in the closed storage cassette. After the biopsies are encased in wax, the storage cassette is opened. The individual storage segments with their sequential step biopsies are separated, sliced, stained and mounted for microscopy while maintaining identification of the biopsies in order of acquisition. This allows precise identification of the extent and location of abnormalities demonstrated by microscopy.

One embodiment of this improved design for serial collection storage and processing is to use the cassette as the site of fixation, processing, staining or tissue testing by loading it with the appropriate reagents.

In another embodiment, the cassette is made of an appropriate material for slicing through the cassette without removing the specimens.

In another embodiment, the storage cassette is used as the reaction chamber for almost in vivo chemical, biological or genetic testing by immediate exposure of the biopsy specimens to reagents before metabolic changes, degradation or contamination can occur.

In another embodiment, the storage cassette is transparent so that the biopsies are immediately available for inspection and analysis without handling. This embodiment avoids the risk of staff exposure to fixatives or infectious agents, yet makes analysis of pristine unfixed specimens by spectroscopy or other physical methods immediately possible without contamination.

In another embodiment, the open tube shaft with a side arm is used for fluid sampling or injection for irrigation, injection of radiopaque contrast or tissue stains through the open biopsy side holes before, during or after biopsy.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a perspective view of the device according to the invention in an open position after obtaining 6 serial step biopsies seen in the individual distal storage segments;

FIG. 2 shows a perspective view of the biopsy and storage cassette in the closed position after six serial step biopsies with the biopsies in situ;

FIG. 3 shows a cross-sectional view of the distal portion of the cassette through the biopsy ports at the beginning of the biopsy sequence with suction applied to pull a knuckle of tissue into the biopsy ports before biopsy and the adjacent storage segment partitions and fluid access perforations;

FIG. 4 shows a cross-sectional view of the distal portion of the cassette through the biopsy ports after cutting of 6 circumferential biopsy specimens that pass into the adjacent storage segments;

FIG. 5 shows the storage and processing cassette after processing to wax and opening with the individual storage segments of serial step biopsies encased in wax in the order of acquisition and ready for slicing;

FIG. 6 shows an embodiment of the invention with a side arm and handle;

FIG. 7 shows the cassette having perforations and a plastic cover over the perforations;

FIG. 8 shows a cross-sectional view of the cassette having ridges and grooves for guiding the biopsies;

FIG. 9 shows the device according to the invention having a screen over the blade; and

FIG. 10 shows the device according to the invention having a spring for maneuvering the cassette against the blade.

### DETAILED DESCRIPTION OF THE PREFERRED EMBODIMENT

For purposes of promoting an understanding of the principles of the invention reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

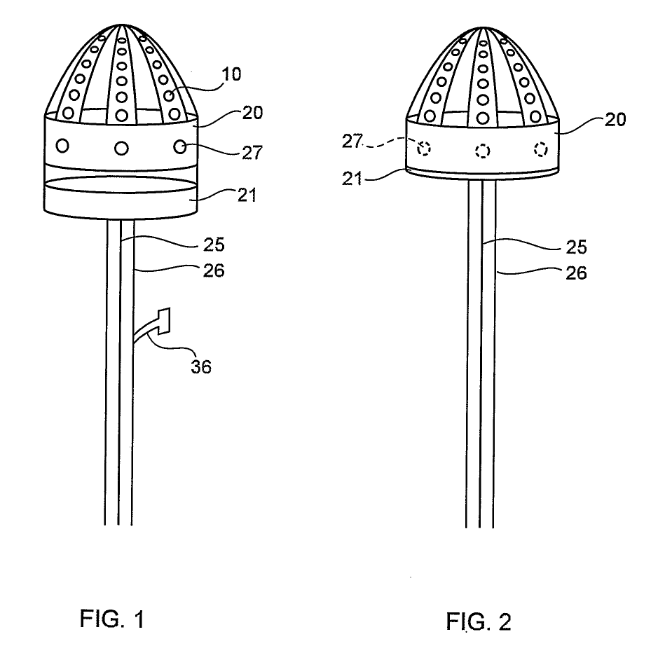

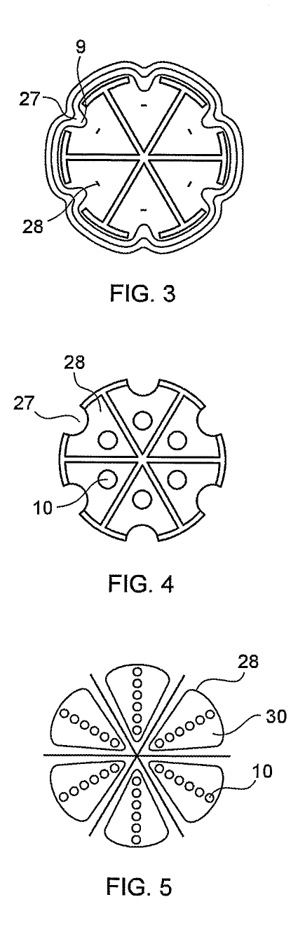

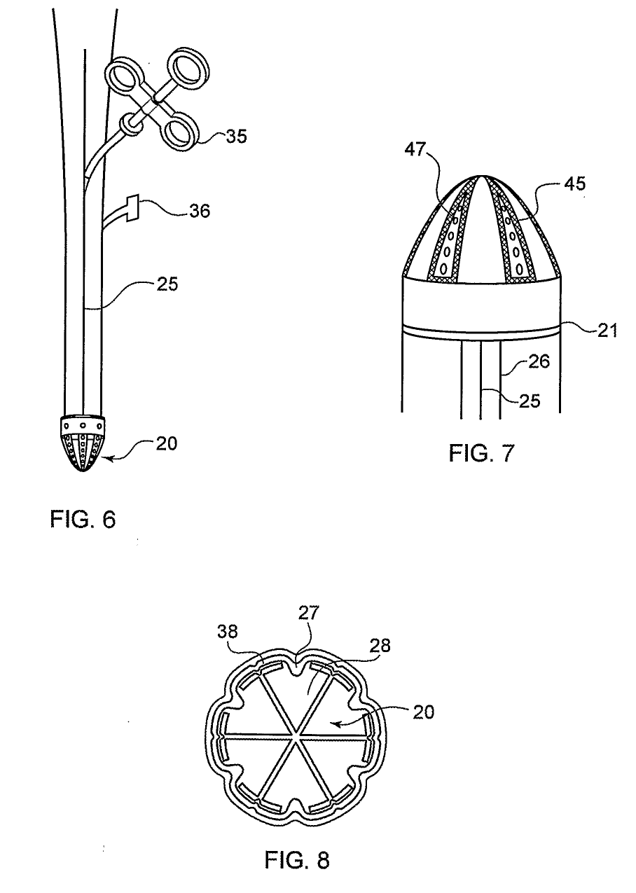

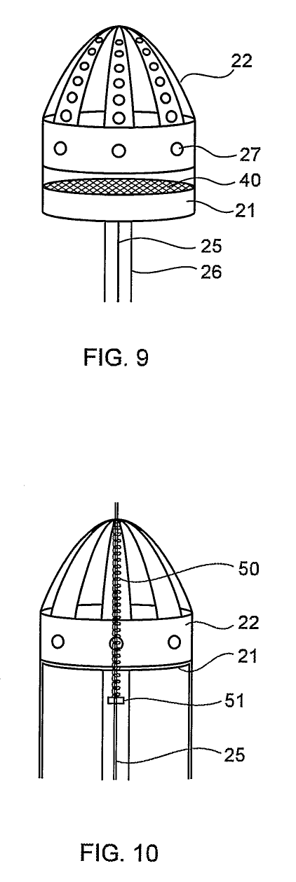

FIGS. 1 and 2 show the device according to the invention, which acquires specimens 10 through a circumferential suction step multibiopsy cutting cassette 20. The blade 21 of the cassette is connected to the central actuator wire 25 inside a relatively large central tube shaft lumen 26 with a side arm 36 for suction or irrigation. The tube shaft 26 is sealed distally to the proximal part of the cassette. Actuator wire 25 is connected to handle 35 (shown in FIG. 6), which seals the lumen 26 to the cassette. In FIG. 1, to prepare for biopsy, blade 21 is pulled proximally by actuator wire 25 to open the biopsy ports 27. In FIG. 3, suction through the side arm and tube shaft pulls the tissue for biopsy 9 into biopsy ports 27. In FIG. 2, moving the actuator wire 25 distally forces the cutting blade 21 over the ports 27 to cut the biopsies 10 and push them into their respective storage chambers 28 as shown in FIG. 4. A screen 40 above the cutting blades, shown in FIG. 9, forces the cut biopsies 10 into their respective storage chambers while maintaining continuity for suction or irrigation through tube shaft lumen 26 and side arm 36. A series of ridges and grooves 38 (See FIG. 8) serves to guide biopsies 10 into their respective storage chambers.

The cassette 20 can be made of any suitable material such as metal or plastic. Guide grooves 38 (FIG. 8) in the cassette prevent twisting to assure that the biopsy ports 27 remain adjacent to their respective storage segments 28. After completion of the biopsy sequence, fixative is injected through the side arm into the processing cassette 20 followed by removal of the tube shaft 26 and actuator wire 25. Alternatively the shaft, wire and plastic cover 47 (FIG. 7) on the perforations 45 may be removed and the entire processing cassette placed in fixative. Alternatively, before biopsy, fixatives and reagents may be placed in the storage segments 28 of the cassette 20 for immediate reaction with the biopsy specimens 10 before metabolic changes occur.

FIG. 5 shows the storage and processing cassette 20 opened after fixation and processing to wax encasement 30 of the specimens in their respective storage segments 28 ready for slicing, slide mounting, wax removal and staining for microscopy in the order of acquisition. A serial number is preferably affixed to each cassette to identify it.

In an alternative embodiment shown in FIG. 10, actuator wire 25 may be connected to the distal biopsy segment 22 that is biased open by an actuator wire spring 50 within the cassette, held by stop 51. (See FIG. 10). Biopsy is then performed by applying suction and then pulling the actuator wire 25 to move the distal segment 22 over the blade 21, closing the cassette 20 and cutting the biopsies 10 from the knuckles of tissue 9 extending into the biopsy ports, in the same way as with the previous embodiment. After completion of the biopsy sequence, the actuator wire 25 is locked at the handle 35 to maintain a closed position. In this embodiment, the biopsy ports 27 are open before biopsy for fluid sampling and injection for irrigation or injection of radiopaque contrast or tissue stains.

Accordingly, while only a few embodiments of the present invention have been shown and described, it is obvious that many changes and modifications may be made thereunto without departing from the spirit and scope of the invention.

1. An apparatus for performing a medical procedure, comprising:
a segmented storage and processing cassette having a blade on one segment extending around a circumference of said cassette, and a plurality of suction biopsy ports located circumferentially around the cassette on another segment of the cassette;
an elongated flexible member connected to said one segment of the cassette and having an aperture extending longitudinally therethrough;
an actuator positioned within the aperture, said actuator being connected to the blade or the cassette, so that movement by the actuator causes the blade to cut biopsies and close the biopsy ports;
a handle for moving the actuator to seal the elongated flexible member;
a tube shaft sealed to the biopsy device and to the handle;
a side arm attached to the tube shaft to permit fluid sampling, suction, irrigation, and injection of tissue stains or radiopaque contrast agents to a biopsy site or into the cassette;
wherein the storage cassette has individual cavities to separate and store cut biopsies of each of suction biopsy port and thereby orient collected biopsy specimens in the cassette.

2. The apparatus of claim 1, wherein the cassette incorporates reagents for chemical, biological or genetic analysis to react with collected biopsy specimens in the cassette before metabolic changes, degradation or contamination can occur.

3. The apparatus of claim 1, wherein the cassette has a set of ridges and grooves to guide relative movement of the biopsies.

4. The apparatus of claim 1, further comprising a screen positioned proximally to blade, to force each series of biopsy specimens into a respective cavity, and to maintain suction and fluid access through the flexible member and to prevent mixing or loss of said biopsies during processing.

5. The apparatus of claim 1, wherein the cassette is perforated to allow entrance of fixative or other reagents without handling and opening of the cassette after processing.

6. The apparatus of claim 5, wherein the perforations are covered with removable plastic for sealing the perforations before use or for removal when said storage cassette has been separated from the flexible member for storage and processing of biopsy specimens.

7. The apparatus of claim 1, wherein the blade is stationary and the actuator is connected to the cassette so that the cassette moves against an opposing spring to cut biopsies while maintaining suction to encourage the entrance of adjacent tissue.

8. The apparatus of claim 1, wherein the biopsy ports are biased open to permit fluid sampling, suction, irrigation, and injection of tissue stains or radiopaque contrast agents into a lumen of the biopsy site before biopsy.

9. The apparatus of claim 1, wherein the cassette is optically transparent.

10. The apparatus according to claim 1, wherein multiple cassettes may be connected to the flexible member.

11. The apparatus according to claim 1, wherein the flexible member is disposable or reusable after washing and disinfecting.

12. The apparatus according to claim 1, wherein the storage cassette is constructed of material to allow cutting without removal of the biopsies.

13. The apparatus according to claim 1, wherein serial number identifies each biopsy cassette.
